(19) United States
(12) Patent Application Publication
(10) Pub. No.: US 2025/0014720 A1
(43) Pub. Date: Jan. 9, 2025

(54) CENTRALIZED MANAGEMENT AND DECENTRALIZED FABRICATION OF CUSTOM MEDICAL DEVICES

(71) Applicant: Sapiyen LLC (DBA Protosthetics), West Fargo, ND (US)

(72) Inventors: Darren JACOBY, West Fargo, ND (US); Leo HAENNI, West Fargo, ND (US); Cooper BIERSCHEID, West Fargo, ND (US)

(73) Assignee: Sapiyen LLC (DBA Protosthetics), West Fargo, ND (US)

(21) Appl. No.: 18/702,163

(22) PCT Filed: Oct. 24, 2022
(86) PCT No.: PCT/US2022/047578

§ 371 Date: Apr. 17, 2024

Related U.S. Application Data
(60) Provisional application No. 63/270,639, filed on Oct. 22, 2021.

(51) Int. Cl. G16H 30/40; B33Y 50/00

(52) U.S. Cl. CPC G16H 30/40; B33Y 50/00

(57) ABSTRACT

Methods for fabricating a custom medical device for an end-user are disclosed. Generally, the present disclosure relates to custom medical devices, and more particularly to three-dimensional modeling of custom medical devices and three-dimensional printing using these three-dimensional models.

### CROSS-REFERENCE TO RELATED APPLICATIONS

This application is related and claims priority to U.S. Provisional Patent Application No. 63/270,639 entitled “Centralized Management and Decentralized Fabrication of Custom Medical Devices” filed Oct. 22, 2021. The entire disclosure of said application is incorporated herein by reference in its entirety.

### BACKGROUND OF THE INVENTION

The present invention generally relates to custom medical devices, and more particularly to three-dimensional modeling of custom medical devices and three-dimensional printing using these three-dimensional models.

Three-dimensional printing for manufacturing custom and semi-custom medical devices continues to grow. For example, many services manufacture prostheses with three-dimensional printers. These three-dimensional printers use three-dimensional printable models in various file formats.

Although the use of three-dimensional printing continues to grow, oftentimes clinicians do not have the training to work with three-dimensional printable models and three-dimension printers. Rather, clinicians have been training for years on how to build custom medical devices, such as prostheses, by hand, but want to get the benefits of three-dimensional printing in-clinic.

### SUMMARY OF THE INVENTION

Methods for fabricating a custom medical device for an end-user are disclosed. An exemplary method includes receiving an identifier of a customer; receiving imaging data corresponding to a desired custom medical device in a first format; converting the imaging data into a set of three-dimensional printer instructions based on the imaging data, a type of three-dimensional printer technology including any specific sub-components, and one or more materials during three-dimensional printing to be used; retrieving a network identifier of at least one three-dimensional printer in a given physical geographic location based on the identifier of the customer, the three-dimensional printer associated with the type of three-dimensional printer technology; sending the set of three-dimensional printer instructions to the at least one three-dimensional printer based on the network identifier; performing during production of the custom medical device: monitoring a video and performance data of the at least one three-dimensional printer while executing the set of three-dimensional printer instructions; identifying any errors in the production of the custom medical device using at least one of the video, status reporting from the at least one three-dimensional printer, or both; and in response to errors being identified, sending a notification to the customer.

The method may include wherein prior to converting the imaging data into the set of three-dimensional printer instructions, further including modifying of the imaging data based on one or more of custom medical devices rules, artificial intelligence and input from a human operator.

The method may further include retrieving, based on the identifier of the customer or the specifications of the desired custom medical device, one or more custom medical device rules; and automatically applying the one or more custom medical device rules during the modifying of the image data.

The method may include wherein prior to sending the set of three-dimensional printer instructions to the three-dimensional printer, further including sending a request to the customer for approval based on at least one of the imaging data or a video recording of a custom model; and receiving approval.

The method may include wherein in response errors being identified, shipping one or more parts for at least one three-dimensional printer.

The method may include wherein in response to errors being identified, dispatching maintenance personnel to service the at least one three-dimensional printer and sharing information for repairing the three-dimensional printer.

The method may further include wherein once the custom medical device of the custom medical device for the customer is completed without errors, automatically receiving imaging data of a custom medical device which has been printed; and comparing the image data of the custom medical device which has been printed with the imaging data corresponding to the desired custom medical device in a first format or a first format which has been modified to identify any differences.

The method may further include storing the differences based on the identifier of the customer in one or more custom medical device rules.

### BRIEF DESCRIPTION OF THE DRAWINGS

The accompanying figures, wherein reference numerals refer to identical or functionally similar elements throughout the separate views, and which together with the detailed description below are incorporated in and form part of the specification, serve to further illustrate various embodiments and to explain various principles and advantages all in accordance with the present invention, in which:

FIG. 1 is a diagram of a decentralized environment with monitoring, according to one example of the present invention;

FIG. 2 is a flow diagram of the process on the centralized computer or server of FIG. 1, according to one example of the present invention;

FIG. 3 is a functional block diagram illustration of a computer hardware platform that can communicate with various networked components, according to one aspect of the present invention; and

FIG. 4 is a simplified diagram of a three-dimensional printer, according to one embodiment of the present invention.

### DETAILED DESCRIPTION

As required, detailed embodiments of the present invention are disclosed herein; however, it is to be understood that the disclosed embodiments are merely examples of the invention, which can be embodied in various forms. Therefore, specific structural and functional details disclosed herein are not to be interpreted as limiting but merely as a basis for the claims and as a representative basis for teaching one skilled in the art to variously employ the present invention in virtually any appropriately detailed structure and function. Further, the terms and phrases used herein are not intended to be limiting; but rather to provide an understandable description of the invention.

### Non-Limiting Definitions

The terms “a,” “an,” and “the” are intended to include the plural forms as well unless the context clearly indicates otherwise.

The phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N, that is to say, any combination of one or more of the elements A, B, . . . or N including any one element alone or in combination with one or more of the other elements which may also include, in combination, additional elements not listed.

The terms “comprises” and/or “comprising,” when used in this specification, specify the presence of stated features, steps, operations, elements, and/or components but do not preclude the presence or addition of one or more other features, integers, steps, operations, elements, components, and/or groups thereof.

The term “configured to” describes the hardware, software, or a combination of hardware and software that is adapted to, set up, arranged, built, composed, constructed, designed, or that has any combination of these characteristics to carry out a given function. The term “adapted to” describes the hardware, software, or a combination of hardware and software that is capable of, able to accommodate, to make, or that is suitable to carry out a given function.

The term “coupled,” as used herein, is defined as “connected,” although not necessarily directly and not necessarily mechanically.

The term “custom medical device” is any device used for medical purposes that may be customized for a specific user or patient and created with a three-dimensional printer. Custom medical device includes Class 1, Class 2, and Class 3 devices. Examples of medical devices include prosthesis, orthopedic casts, prosthetic ears, cranial helmets, scoliosis braces, transplantable organs, skin, and other printable medical devices.

The term “custom medical device rule” is any rule that is applied by a human or automatically by software when modifying the image data. For example, a custom medical device rule may include modifications to physical dimensions, geometries, printer selection, and other specifications based on inputs.

The terms “including” and “having,” as used herein, are defined as comprising (i.e., open language).

The term “prosthesis” refers to an artificial device that replaces or augments the missing or impaired part of an animal body. A prosthesis can be used to partially or fully restore lost functionality. It can also be used for an externally applied device, or orthosis, in order to stabilize or support an injured or naturally deficient body part to allow more effective healing or guide the functional or developmental rehabilitation of a body part over time.

The term “three-dimensional editing software” means software that can manipulate three-dimensional printable models that may be read in various file formats and output content adapted to specific requirements for different three-dimensional printer technology types. Examples of three-dimensional editing software include Autodesk Fusion360, AutoCAD, and others.

The term “three-dimensional printable models” means any model that can be processed into instructions usable by any type of three-dimensional printer technology. Three-dimensional printable models may be created with a computer-aided design (CAD) package via a three-dimensional (3D) scanner or by a plain digital camera and photogrammetry software. The three-dimensional printable models may be stored in a stereolithography file format (STL), an additive manufacturing file format (AMF), and other formats.

The term “type of three-dimensional printer technology” means any additive manufacturing process used in the construction of three-dimensional objects, including fused deposition modeling (FDM), frostereolithography (SLA), selective laser sintering (SLS), powder bed fusion, selective laser melting (SLM), electronic beam melting (EBM), direct energy deposition, and three-dimensional bioprinting. Components of a type of three-dimensional printer technology include a tool-head, a nozzle, and heater cores.

The term “three-dimensional printer programming language” means any programming language, such as G-code, that instructs three-dimension printers and other additive manufacturing processes.

The term “three-dimensional scan software” means any software and hardware capable of creating a 3D image of a physical object. Providers include the Scan CapteviaPlus application from RODIN SAS, Merignac, France; software solutions for acquisition and 3D measurements of the human body from TechMed 3D Affiliates, Quebec, Canada; Comb software from Comb O&P Chardon, OH, USA; OMEGA® Scanner 3D from WillowWood Global LLC, Mt. Sterling, Ohio, USA; Structure Sensor by Occipital; and others.

### Network Topology

The present invention offers a managed network of distributed decentralized three-dimensional (3D) printers. Elements or nodes of the network include client computers, scanners, server or web servers, management consoles, and three-dimensional printers, all communicatively coupled over a global communication network, such as the internet. The network functions by having centralized management of the network of printers deployed at customer sites around the country and/or the world. In one example embodiment, these printers can be deployed in standalone booths, such as a kiosk. These printers can be of any type of three-dimensional printer technology (e.g., FDM, DLP, SLS, etc.) but are all consistent in being connected via the internet to the managed network of three-dimensional printers at various geographic locations.

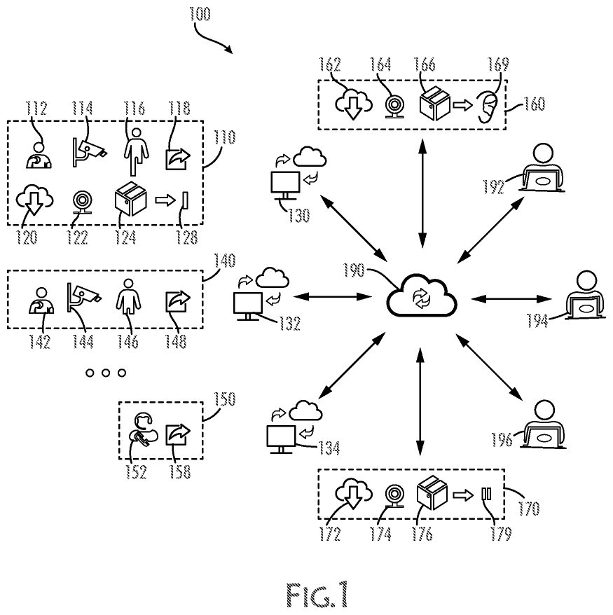

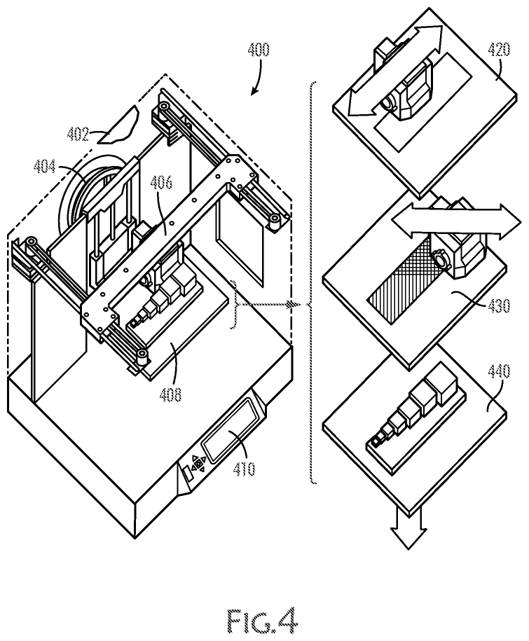

Turning now to FIG. 1 is a diagram 100 of a decentralized environment with monitoring, according to one example of the present invention. Shown are customer locations or nodes 110, 140, 150, and three-dimensional printing nodes or locations 160 and 170. Also shown are customer service technicians 192, 194, 196, all communicating over a global communications network 130, 132, 134, 162, 172 with a centralized server or computer or cloud computing service 190. In some example embodiments, centralized server or computer or cloud computing service 190 can be employed as a central node, management node, or interoperability hub. In some instances, for example, a central node, management node, and/or interoperability hub may run one or more applications and/and incorporate a number of components and/or engines to monitor and manage a set of distributed decentralized three-dimensional (3D) printers. In some embodiments the monitoring may be carried out by a central node (e.g. node 190 of FIG. 1) and/or a centralized management system (e.g. centralized management system 840 of FIG. 4). In some embodiments, a central node and/or centralized management system may have implemented thereon or otherwise access one or more datasets for the coordination of a plurality of disparate or incompatible nodes. For example, a central node may convert or compile datasets using one or more agents.

Each customer location 110, 140 may have different customers 112, 142 that use various types of three-dimensional scan software 114, 144 and equipment to imaging capture corresponding to a desired custom medical device of end-user 116, 146. This three-dimensional scan software can capture the three-dimensional scans in a variety of file formats 118, 148. In addition to three-dimensional scan data transmitted in one or more file formats, three-dimensional scans may incorporate or otherwise be associated with additional data and/or information files. In some alternative example embodiments, customer 152 is telephoning, faxing, emailing, or texting in requirements 158 for a desired custom medical device. In one example embodiment, there may be multiple customers per customer location, e.g., five practitioners at one company, each with different preferences.

Customer location 110, 140 also has at least one three-dimensional printer 124 that is connected to the network or cloud 120. It will be appreciated that each customer location 110, 140 can be connected to a network (e.g. network 190) or sub-network, for example a local-area network. The three-dimensional printer can be a variety of types of three-dimensional printer technology. It will be appreciated that a three-dimensional printer at any location 110, 140 can further be associated with a data set from one or more data stores in logical connection with a distributed network. The three-dimension printer is typically monitored by a camera 122 and other sensors and is capable of producing or otherwise fabricating a prosthetic 128 or medically implemented part.

In some embodiments, customer locations 140 and 150 do not have a three-dimensional printer on-premise. Rather, they receive output information and/or data from three-dimensional printers that are offsite 160 and 170. The technology type of the three-dimensional printer, along with availability information and geographic location information, will be utilized at least in part to determine which printer to select. Note that each of the printer nodes 160 and 170 is coupled to a distributed network, for instance via cloud 162 and 172 and have cameras 164, 174 to monitor output from three-dimensional printers 166 and 176, producing or otherwise fabricating customizable medical devices, such as prosthetic ear 169 and/or prosthetic legs 179. In one example, printer nodes 160, 170 can be arranged as, or in the form of, a customer kiosk. In a self-help kiosk, other functions may be included. One such function is a device, such as a conveyor or robotic arm, to move the custom medical device from the print bed area to a holding or storage area, once the printing has completed. Other items available at the kiosk could include grinders, Dremmel® tools, or files to allow any extra support materials to be removed. Vises, clamps, and other fixturing could also be present to hold a custom medical device while extra support material is removed.

In another example, the kiosk includes an interactive video screen with instructional videos and content on the kiosk that will guide the user in how to take any next steps with the product. These can be intelligently shown based on the product that is printed. A video screen can also be used for feedback on the product to improve future product quality, to allow the customer to order additional materials, or to control the printer functionality in a variety of ways.

The customer service technicians 192, 194, 196, in one example, may edit or create a three-dimensional printable model using three-dimensional editing software as further described below.

### System Architecture Flow

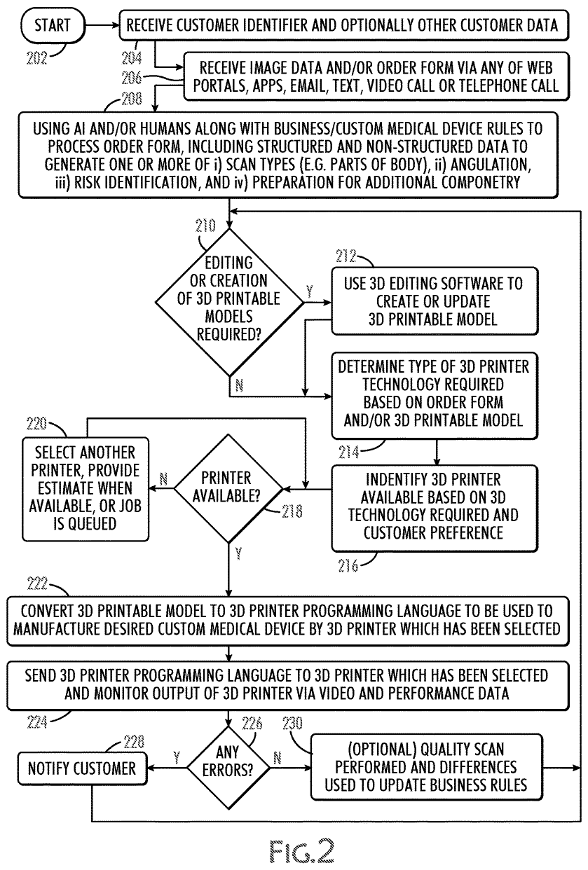

FIG. 2 is a flow diagram of one or more processes carried out by the centralized computer or server of FIG. 1, according to one example embodiment of the present technology. The process starts at step 202 and immediately proceeds to step 204.

At step 204, a customer identifier, such as logon or account name, is received as input data. This may be, for example, received through a smartphone application (app), over the telephone, or through a web portal as further described below in the sections entitled “Requests Received By Centralized Server” and “Communications.” The process continues to step 206.

At step 206, image data and/or order (form) information or set of instructions is received. As with a customer identifier, this image data information can be received via any one of web portals, apps, email, text, video call or telephone call.

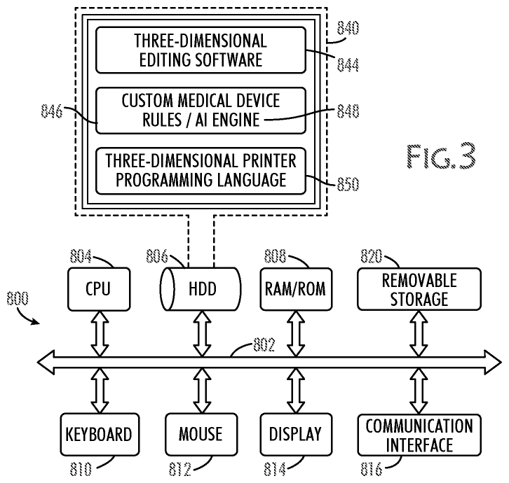

At step 208, using AI (e.g. AI 848 of FIG. 3) and described above, and/or humans 192, 194, 196 along with business/custom medical device rules 846 (e.g. stored in connection with centralized server and/or management node 190 of FIG. 1) to process an order form, including structured and non-structured data. In some embodiments, order information may be parsed by one or more nodes of the system. This produces or otherwise generates one or more of: i) identification of scan type(s) (e.g., parts of the body), ii) angulation, iii) risk identification, and iv) preparation for additional componentry, as described above. In some embodiments, data from this step may be collected into a processed order data set. Further details of step 208 are described in the section below entitled “Order Clarification and Validation.” The process continues to step 210.

At step 210, a test is made to determine whether editing or creation of 3D printable models is required. This can be determined through a combination of the order itself 118, 148, 158, customer feedback, business rules/medical device rules 846, AI 848, and customer service operators 192, 194, 196. In some instances, a test can comprise a query of any number of databases or datasets as described above. In some instances, the test may be automatically implemented. If edits are determined to be necessary, the process continues to step 212. Otherwise, the process continues to step 214.

At step 212, three-dimensional editing software driven by a user, business rules, or generative design, is used to create or update the three-dimensional printable model, and then continues to step 214. In some embodiments, the updating is implemented in part by a centralized server and/or management node. For example, in some embodiments, a management node may query one or more stored rules and automatically update the three-dimensional printable model.

At step 214, a type of three-dimensional printer technology is determined based on order form (or order information) and/or three-dimensional printable model. For example, the various technology types include deposition modeling (FDM), frostereolithography (SLA), selective laser sintering (SLS), fused metal deposition, and/or three-dimensional bioprinting. The process continues to step 216.

In step 216, a search is carried out, for example by node 190, to identify a three-dimensional printer based on customer preference, and/or geographical location, and other criteria, such as, for example:
1) the availability of the three-dimensional printer;
2) no product on the printer bed;
3) is not printing a custom medical device;
4) printer queue is scheduled to print a custom medical device; or
5) time remaining to complete another custom medical device.

Optionally, a notification process for local printer maintenance may be used. In this example, the three-dimensional printer is monitored 122, 164, 174. Centralized management system 840 for instance can notify people working at the node of printer maintenance required. Examples would be that AI monitoring the camera feed that shows the print bed and identifies that the previous custom medical device has not been taken off the print bed. In this example, the centralized management system 840 automatically (or via a user or user device) notifies the printer node or local operator to remove the print on the bed before the next print job can begin. The process continues to step 218.

At step 218, a test (e.g. an automated query to one or more of the identified three-dimensional printers) is made to see if the identified three-dimensional printer based on step 214 and step 216 is available. In the case the three-dimensional printer that was identified is not available, another three-dimensional printer is selected, an estimate on availability is provided, or the print job is queued in step 220 and continues to step 218 when the printer becomes available. Otherwise, the process continues to step 222.

At step 222, the three-dimensional printable model is converted to a three-dimensional printer programming language to manufacture the desired custom medical device by a three-dimensional printer which has been identified in steps 214 through 218. Three-dimensional programming language is created with settings and materials determined based on the type of product/order form/customer preference/AI. The process continues to step 224.

At step 224, the three-dimensional printer programming language is sent to the three-dimensional printer, which has been selected. The output of the three-dimensional printer is monitored via video 122, 164, 174, and other sensors and the three-dimensional printer's performance and reporting data. Optionally, the customer/user can start the printer themselves, or the customer service/AI/business rules can start the print on the customer's behalf. Further details of step 224 are described in the section below entitled “Deployment.” The process continues to step 226.

At step 226, a test is made to determine if there were any errors during the three-dimensional printing process, which were identified in step 224. In the case there are errors identified, the customer is notified at step 228, and the process returns to step 210. In one example, the process automatically returns to the design process with new or updated instructions in step 210 and the process continues. One example of an error is based on the video feed of the three-dimensional printing, recognizing when a print job is failing before it completes. In this example, the system may proactively cancel the print job through monitoring software prior to completion. This step may include a further step of remotely troubleshooting the printing device before the customer is notified to determine if the error can be resolved remotely. If not, the customer may be notified of how to resolve the error with assistance. If the error still cannot be resolved, a field team may be dispatched to resolve the error on site, or replacement parts may be sent. In the case of no errors, the process continues to step 230.

Step 230 is an optional step in which a quality scan of the customer medical device that has been fabricated is compared with the original scan received. Any differences that are identified may be used to update business rules. Additionally, at the end of every print the customer is automatically surveyed on the quality of the print and any differences/feedback gleaned from such survey is used to update business rules and AI. The process returns to step 210.

One type of notification may be automated shipping of replacement consumables and/or parts for the three-dimensional printer. For example, if a printer node is running out of consumable print material, the centralized management system 840 automatically ships the consumable material. More specifically, materials, such as, filament rolls with identifying tags, either an NFC tag or a barcode, is confirmed at the printer node with a near-field communications or scanner 122, 164, 174, and registers the material being used at each printer 124, 166, 176. It is important that filaments are checked in and out, as different types of filaments (materials, colors), can be swapped between prints. Syncing this scanning with print monitoring ensures the centralized management system 840 tracks the quantity and type filament consumed to build each custom medical device. This permits a real-time filament inventory on a per printer and per customer basis. Tracking this will allow the centralized management system 840 to automatically place an order on behalf of customers to replace filament and have it ship before a prediction that will run out of a certain material or color.

This inventory information, predictive analytics, automated order placement, billing, and fulfillment, will all happen on the backend of the platform and notify the customer that the filament is shipping or ask for approval for order placement dependent on user settings. In addition, monitoring of the nozzle component having certain componentry in the three-dimensional printer at a specific printer node can be done to indicate upcoming possible failure. For example, lower print speeds may be interpreted that a print nozzle is partially clogged. Further, in another embodiment, the three-dimensional printer is capable of automatically swapping filaments based on input from the centralized management system or based on the G-codeit receives. Still in another example, the three-dimensional printer is able to swap hardware, such as, changing out different sized or damaged nozzles, based on input form the centralized management system or based on G-code. The centralized management system 840 automatically, or with user input or user device generated input, ships a new nozzle to this printer node.

The centralized management system 840 monitors each printer node 110, 160, 170 by reviewing performance data and running predictive analytics, which in some embodiments is carried out in real-time. This interfaces with order placement, fulfillment, and billing, creates seamless proactive maintenance of three-dimensional printers. It will be appreciated that centralized management system 840 may be implemented as a central node (e.g. node 190 of FIG. 1) or distributed across multiple nodes and/or computing devices.

Feedback on the print, such as to enable nodes to give feedback to the centralized management server on the quality and accuracy of the custom medical device manufactured, includes allowing this as the feedback of the node to the central system after the custom medical device has been deployed.

### Requests Received By Centralized Server

Types of orders and requests from customers 112, 142, 152 to the centralized management server 190 may be performed using client computers, client portals via a web server, or nodes on the network. They participate in the managed three-dimensional printers by submitting to the centralized management server. Any one or more of the following types of requests may be used in producing a custom medical device at one of the three-dimensional printers 124, 166, 176 located in various geographic locations or nodes:
1) A scan 118, typically produced by three-dimensional scan software that has been modified by the customer at a client system and only needs to be converted into the proper format, such as a three-dimensional printable model, for decentralized manufacturing at one of the three-dimensional printers;
2) A scan 148, typically produced by three-dimensional scan software that has not been modified but needs to be modified, such as by using three-dimensional editing software, before being converted into the proper format, such as three-dimensional printable models, and decentralized manufactured at one of the three-dimensional printers; and
3) A set of measurements or general description 158 of the desired custom medical device, sent to the centralized management server for design of the custom medical device, such as, by using three-dimensional editing software, before being converted into the proper format, such as three-dimensional printable models, and followed by decentralized manufacturing at one of the three-dimensional printers.

The customer sending the data to the centralized management server can request that the custom medical device be manufactured at any of the printers under that customer's control or at a centralized printer if necessary/requested. For example, if customer 112 owns three-dimensional printer locations 110 and 160, customer 112 can request that the custom medical device be printed at any, some, or all of the three-dimensional printer locations 110 and 160.

### Communications

Methods for communicating custom medical device information: As a customer, the customer can send information for manufacture in any or using multiple of the following methods:
1) Through submission in the customer ordering platform (centralized management system 840 of FIG. 3) on centralized management server's 190, which can take any of the formats requested above. This platform can be accessed by any/all of the following:
   a) On the screen that may or may not be attached to the three-dimensional printer;
   b) Through mobile devices, whether on an application or through the web; and
   c) Through web on a desktop computer.
2) Through electronic means with unstructured instructions or by attaching a finished or to-be-modified scan, for example, over email, text, etc.; and
3) Through direct contact with the centralized management server 190, over the phone or video conference, describing the requirements and potentially supported by means 1 & 2.

### Order Clarification and Validation

A process of order clarification and validation is now described. Once an order is received by the centralized management server 190, the customer service representatives 192 to 196 may clarify the needs with the customer via any, multiple, or all of the methods by which the customer can submit the order. The method that the order was submitted does not have to be the same method by which the clarification occurs. The order can also be validated before final manufacturing, whereby the centralized management server will receive final approval by the customer before deploying for manufacturing. The validation need not always occur and can be used on a per customer, per custom medical device type, or per type of request.

Next, a process of order modification and conversion takes place. Once the submission has been received or clarified (if required), the centralized management server 190 will go ahead and make the design and/or modification based on the request received. The centralized management server 190 can use one, some, or all of the following means to make designs and/or modifications to reach a final custom medical device:
1) Artificial intelligence, in one example, is deployed to take the data inputs provided by the customer and generate modifications on an existing design or a newly generated design based on the customer's set of requirements. These artificial intelligence methods can be proprietary methods or use a technology provided by a third party. These methods use learnings from previous design processes and training of the artificial intelligence models to generate a probabilistic change in the design and/or modifications;
2) Custom Medical Device Rule or Business Rules methods: These methods are repeatable and stochastic changes that occur to the design and/or modifications of the submitted request, based on data about the submitting party or the custom medical device requirements. For example, if the submission is for provider Y, the business rules can automatically set the width of the custom medical device to be less than Z cm, as provider Y has indicated s/he would always like his/her custom medical devices to be less than Z cm wide. In addition, the rules can be applied as a custom medical device feature request that can go across the network—for example, in any case, where custom medical device type XY is requested from any provider, the business rules will modify the height of the custom medical device design to be less than or equal to YZ inches, as all XY custom medical devices should be less than YZ inches; and
3) Human-driven modifications—based on submitted information and/or clarification between the submitter and the centralized management server, a human can make direct design choices or modifications to the custom medical device design choices. This human-driven modification can use the assistance of software that makes the modification process easier, or completely “free-hand” in accordance with the customer's wishes.

Once designs (if required), modifications (if required), and validations (if required) have been achieved for the custom medical device, the centralized management server 190 will convert the design into a printable format that the desired decentralized printer node supports—this can vary widely based on the printer technology, make of printer, material use, and desired end custom medical device. This conversion process can be completed with the same automation processes described for modifications or by human intervention, as shown in the section above entitled “Human-driven modifications.”

When submitting a first form scan, the user will submit an accompanying order form consisting of structured and unstructured data. The required and optional fields are populated based on custom medical device type selection. In addition to these fields, the user will be able to submit unstructured data in the form of text, photos, or video. Furthermore, the user can request a call/videoconference with the central node during the process to add any more details around the specifications or requirements.

With regards to completing the order form, image analysis using artificial intelligence algorithms can be used to help select and prepopulate the order form. For example, in the case of a scan of a transfemoral amputated residual limb, the artificial intelligence algorithm can identify the nature of the limb, and auto-select the type of form to be filled out—in this case, an above-knee prosthetic socket. The methods for this type of analysis will be described below.

Once the image and the accompanying order form have been sent to the central node, the image and the order form can be processed by a combination of the three methods (human, custom medical device rules, AI). In terms of artificial intelligence, the server will do the following things:
1) Interpret Unstructured Data—use a combination of NLP, video recognition, and speech recognition to create additional structured commands for implementation in the design changes;
2) Identify Scan Type—using image recognition built on a variety of standard AI models. The AI will identify what part of the body is being represented in the scan. e.g., foot, head, transtibial amputated residual limb, transfemoral amputated residual limb;
3) Identify Key Points—once the body part has been identified, the AI can identify key points on the limb for the purpose of further modifications and validation, e.g., on the transtibial amputated residual limb, identify the patella, fibular head, posterior side, anterior side, tibial crest, distal tibia, and distal fibula;
4) Identify Angulation—based on the scan of the body part, use image recognition to understand the current angulation of the body part. For example, if the limb is in a flexed, extended, adducted, or abducted position, and the degree to which it is angled;
5) Risk Identification—based on the scan of the body part, use image recognition to identify areas of concern on the patient's limb, e.g., bruises, callouses, open wounds;
6) Generative Design—based on the desired changes indicated in the order form, and the data identified using artificial intelligence, custom medical device rules will be used to implement design changes in the desired areas. For example, if the user requests changes in thickness, relief or buildup at certain points (automatically placed due to functionality above entitled “Identify Scan Type” and/or “Identify Angulation”, changes of angulation (absolute or in relation to identified angulation in point 4);
7) Merge Multiple Designs—based on the desired changes indicated in the order form, the supplied image and templates can be merged together to create a final custom medical device. For example, if a transtibial limb is shared with the request for a transtibial socket to be assigned for use with a pin-lock adapter, the AI can use identified points to pick the right area for placement in the section entitled “Identify Key Points”, the custom medical device rules can pull the requested attachment from a server, and the AI can merge the two designs into a final custom medical device; and
8) Preparation For Additional Componentry—based on the desired changes indicated in the order form and the identified device type, the design can be modified by algorithms to be able to pair with additional components. For example, if a wrist orthosis is to be paired with a certain strap in post-processing, the space for the strap to be added can be automatically placed in the design in the point, set by custom medical device rules, and chosen based on identified points.

### Deployment

Deployment: Once the digital file has been converted to the proper format for manufacturing at the desired printer node(s), the custom medical device can be deployed and manufactured by the following means:
1) Automatically deployed through the software management tool the centralized management server uses to manage the deployed devices. This will be configured by having the centralized management server software system be connected to the deployed printers through API calls and connections over the backend to the node's printer via the printer's internet connection;
2) By manual connection to and uploading of the scan to the 3D printer. This can be achieved by establishing remote access to the printer via VPN and directly uploading the design file to the end printer node; and
3) By sharing the file with the local customer for local deployment—in cases where the internet connection between the centralized management server and the printer has failed, the centralized management server can share the file with a person at the desired node, who can manually upload the converted print to the printer for printing. This file can be shared by directly emailing the local customer or by making the file available in the ordering portal.

All of this occurs in parallel, with the centralized management server supporting a distributed set of customers, printer nodes, and custom medical device requests.

For scanning existing devices and printers (even if the devices are made elsewhere via another technology) scanning technology can be embedded into the three-dimensional printer or kiosk the printer is housed in itself. In this example, embedding scanning technology into the three-dimensional printer permits any adjustments made to the shape in-clinic or to a device manufactured elsewhere or by other means, can be easily and accurately communicated to the central company. Technology here could be a single physical scanning probe embedded into the kiosk that touches the device or an array of cameras placed in the kiosk that photograph the shape from multiple angles and digitally reconstruct a 3D surface from the images, or embedding any of the scan technologies previously described. Other scanning technology can be included as follows:
1) Integration of a digital glass sensor for taking foot shapes for the generations of scans for orthotics—Many existing technologies for example Sendor Product, Inc and Delcam iQube Scanner, but the unique concept herein is to include this technology in a retractable shelf in the kiosk, allowing patients to be scanned within the device, and automatically have that sent to the platform for further modification or automated orthotic printing. The user will also have an interface to add details about the patient when the scan is being performed in the device.
2) Skin tone matching—The kiosk can have an embedded camera that can be used to identify the skin tone of the user of the end custom medical device in order that the correct filament can be suggested and used for manufacturing the device. This information can also be stored for the end-user the custom medical device is being manufactured for through connection to the backend platform for future manufacturing.
3) Weight-bearing scanning sleeve—To enable the seamless scanning of a residual limb under weight-bearing conditions, the end-user can utilize the proprietary scanning sleeve. The sleeve will be a silicone-based sleeve that wraps around the residual limb and then has the user stand with the residual limb under load, in either a box of sand or in a generally usable socket that can be tightened to create pressure. The sleeve will have electronic sensors that make a three-dimensional printable model of the residual limb under load, allowing for the most accurate model of the leg that creates the basis for the most accurate device. This three-dimensional model can then be communicated to the central platform via connection to the kiosk internet connection or an application on a mobile device with the connection to the central platform. The device can then be modified and deployed for manufacturing using the same method above.
4) Weight-bearing scanning solution—In order to enable the seamless scanning of a residual limb under weight-bearing conditions, the end-user can utilize the proprietary scanning solution. This would be an aqueous or mineral-based (sand) solution that the end user places the residual limb in, displacing the solution. The solution can then have a current run through it or a radar scan of the entire solution that shows the displaced area and creates a 3D shape of the limb. This 3D shape can then be communicated to the central platform via connection to the kiosk internet connection or an application on a mobile device with a connection to the central platform. The device can then be modified and deployed for manufacturing using the same method above.
   Example 3D Printer and/or System

FIG. 4 is simplified diagram 400 of a three-dimensional printer, according to one embodiment of the present invention. An outer box 402 holds the other components of the three-dimensional printer 400. Material 404, such as plastic or synthetic material, is held in a spool, in the example for printing the three-dimensional object. A robotic print head and extruder 406 prints custom medical devices by layering material on a surface, such as in one example, through a hot-glue gun. A mobile plate 408 moves vertically as each layer of the custom medical device is printed. Local controls/display 410 enables any local controls as necessary.

The process of printing a customer medical device begins with the print head making an outline of the custom medical device on the surface of the build plate in step 420. Next is step 430, after the outline is made, the shape is filled in. Fill-in patterns vary depending on the custom medical device, but cross-hatch patterns are the most common. Next, in step 440, the build plate is moved down as a new layer is outlined and filled until the custom medical device is completed.

### Example Computer System or Framework

FIG. 3 is a functional block diagram illustration of a computer hardware platform that can communicate with various networked components, according to one aspect of the present invention. In particular, FIG. 3 illustrates a particularly configured network or host computer platform 800, as may be used to implement the method in FIG. 1 and FIG. 2 above. FIG. 3 can implement any or part of systems 110, 140, 150, 160, and 170.

The computer platform 800 may include a central processing unit (CPU) 804, a hard disk drive (HDD) 806, random access memory (RAM) and/or read-only memory (ROM) 808, a keyboard 810, a mouse 812, a display 814, and a communication interface 816, which are connected to a system bus 802. The HDD 806 can include data stores.

In one embodiment, the HDD 806, has capabilities that include storing a program 840 that can execute various processes, such as, for executing customer ordering platform with a three-dimensional editing software 844, customer medical device rules 846 and/or AI Engine 848 and three-dimensional printer programming language 850.

In one embodiment, a program, such as Apache™, can be stored for operating the system as a Web server. In one embodiment, the HDD 806 can store an executing application that includes one or more library software modules, such as those for the Java™ Runtime Environment program for realizing a JVM (Java™ virtual machine).

As will be appreciated by one skilled in the art, aspects of the present invention may be embodied as a system, method, or computer program product, such as a removable storage 820 at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

### Examples

The flowchart and block diagrams in FIG. 1 through FIG. 4 illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The description of the present application has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application and to enable others of ordinary skill in the art to understand various embodiments of the present invention, with various modifications as are suited to the particular use contemplated.

1. A method for fabricating a custom medical device for an end-user, the method comprising:
receiving an identifier of a customer;
receiving imaging data corresponding to a desired custom medical device in a first format;
converting the imaging data into a set of three-dimensional printer instructions based on the imaging data, a type of three-dimensional printer technology including any specific sub-components, and one or more materials during three-dimensional printing to be used;
retrieving a network identifier of at least one three-dimensional printer in a given physical geographic location based on the identifier of the customer, the three-dimensional printer associated with the type of three-dimensional printer technology;
sending the set of three-dimensional printer instructions to the at least one three-dimensional printer based on the network identifier;
performing during production of the custom medical device:
monitoring a video and performance data of the at least one three-dimensional printer while executing the set of three-dimensional printer instructions;
identifying any errors in the production of the custom medical device using at least one of the video, status reporting from the at least one three-dimensional printer, or both; and
in response to errors being identified, sending a notification to the customer.

2. The method of claim 1, wherein prior to converting the imaging data into the set of three-dimensional printer instructions, further comprises modifying of the imaging data based on one or more of custom medical devices rules, artificial intelligence and input from a human operator.

3. The method of claim 2, further comprising:
retrieving, based on the identifier of the customer or the specifications of the desired custom medical device, one or more custom medical device rules; and
automatically applying the one or more custom medical device rules during the modifying of the image data.

4. The method of claim 1, wherein prior to sending the set of three-dimensional printer instructions to the three-dimensional printer further comprises:
sending a request to the customer for approval based on at least one of the imaging data or a video recording of a custom model; and
receiving approval.

5. The method of claim 1, wherein in response errors being identified, shipping one or more parts for at least one three-dimensional printer.

6. The method of claim 1, wherein in response to errors being identified, dispatching maintenance personnel to service the at least one three-dimensional printer and sharing information for repairing the three-dimensional printer.

7. The method of claim 1, further comprising:
once the custom medical device of the custom medical device for the customer is completed without errors, automatically receiving imaging data of a custom medical device which has been printed; and comparing the image data of the custom medical device which has been printed with the imaging data corresponding to the desired custom medical device in a first format or a first format which has been modified to identify any differences.

8. The method of claim 7, further comprising:
storing the differences based on the identifier of the customer in one or more custom medical device rules.
